(19) United States
(12) Patent Application Publication
(10) Pub. No.: US 2024/0383076 A1
(43) Pub. Date: Nov. 21, 2024

(54) METHOD FOR DETERMINING THE FILLING WELDING PARAMETERS OF LARGE DEFORMATION PIPELINE STEEL BASED ON SECONDARY REGULATION METHOD

(71) Applicant: Tianjin University, Tianjin (CN)

(72) Inventors: Yongdian HAN, Tianjin (CN); Jiyuan FEI, Tianjin (CN); Lianyong XU, Tianjin (CN); Lei ZHAO, Tianjin (CN); Kangda HAO, Tianjin (CN); Hongyang JIN, Tianjin (CN)

(73) Assignee: Tianjin University, Tianjin (CN)

(21) Appl. No.: 18/199,371

(22) Filed: May 19, 2023

(51) Int. Cl. B23K 31/12; G01N 3/08; G01N 33/207

(52) U.S. Cl. CPC B23K 31/12; G01N 3/08; G01N 33/207; G01N 2203/0017; G01N 2203/0067; G01N 2203/0298

(57) ABSTRACT

A method for determining the filling welding parameters of large deformation pipeline steel based on secondary regulation method includes: welding specimens to be welded for secondary welding thermal simulation experiments based on a thermal simulation to obtain samples after thermal simulation; processing the samples after thermal simulation into CTOD samples and calculating fracture toughness parameters; pre-loading of specimens requiring pre-strain after thermal simulation by uniaxial tension, and then processing samples before and after pre-strain after thermal simulation, conducting slow strain rate tension tests and calculating stress corrosion cracking susceptibility parameters; comparing the change in elongation of the samples before and after pre-strain and calculating the pre-strain sensitivity parameters; determining secondary thermal simulation parameters; converting the secondary thermal simulation parameters into welding heat input parameters; determining welding parameters based on welding heat input parameters; determining the optimal role of the welding parameters.

### TECHNICAL FIELD

The present application relates to the field of welding technology and specigically relates to a method for determining the filling welding parameters of large deformation pipeline steel based on secondary regulation method.

### BACKGROUND

In the deep—sea oil production engineering environment, Reel-lay is a promising and efficient solution for pipe laying. However, the pipeline will be subjected to reciprocating extreme loads during laying, which puts forward higher requirements for the toughness of the material. On the other hand, more and more oil and gas resources contain hydrogen sulfide. The acidic medium containing hydrogen sulfide will cause corrosion and stress corrosion of pipelines, and may lead to serious safety problems. The welded pipe is an important processing link for pipeline preparation. In the multi-layer multi-pass welding, the weld of the previous pass will be subjected to the heat effect of the next pass, forming a complex heat-affected zone. The critical coarse grain zone is the weakest area in the whole welded joint, and its toughness and stress corrosion resistance are significantly reduced compared with the base metal.

Therefore, it is necessary to develop and design a multi-layer multi-pass welding process with high performance, to achieve the fracture toughness of the critical reheat coarse grain zone in the welded heat-affected zone under large deformation conditions and the synergistic enhancement of the resistance to sulphur stress corrosion cracking to ensure the safety of the pipeline steel in industrial practical applications.

### SUMMARY

In view of this, embodiments of the present application provide a method for a determining the filling welding parameters of large deformation pipeline steel based on secondary regulation method, to achieve synergistic resistance to sulfide stress corrosion cracking and fracture toughness in the critical reheated coarse grain zone of the heat-affected zone of the welded pipeline steel under pre-strain.

In order to solve the above technical problems, this embodiment of the specification embodiment is implemented in the following way:
the embodiment of the present specification provides a method for determining the filling welding parameters of large deformation pipeline steel based on secondary regulation method, comprising:
welding specimens to be welded for secondary welding thermal simulation experiments based on a thermal simulation to obtain samples after thermal simulation;
processing the samples after thermal simulation into Crack-tip Opening Displacement (CTOD) samples and calculating fracture toughness parameters;
pre-loading of specimens requiring pre-strain after thermal simulation by uniaxial tension, and then processing samples before and after pre-strain after thermal simulation, conducting slow strain rate tension tests and calculating stress corrosion cracking susceptibility parameters,
comparing the change in elongation of the samples before and after pre-strain and calculating the pre-strain sensitivity parameters;
analyzing in a comprehensive manner the determination of secondary thermal simulation parameters by combining pre-strain sensitivity parameters, fracture toughness parameters and stress corrosion cracking susceptibility parameters;
converting the determined the secondary thermal simulation parameters into welding heat input parameters by calculation in accordance with the three-dimensional heat transfer formula;
determining welding parameters based on welding heat input parameters;
determining the optimal role of this parameter by comparing the welding parameters with the conventional welding parameters of the sulfide stress corrosion cracking stress intensity factor.

Optionally, the samples to be welded are multiple, and multiple samples to be welded have different cooling rates for the secondary heat cycle.

Optionally, the fracture toughness parameters include CTOD value, and the calculation formula of fracture toughness parameters is:

$$f ( \frac{{a}_{0}}{W} ) = \frac{3 {( \frac{{a}_{0}}{W} )}^{0.5} [ 1.99 - ( \frac{{a}_{0}}{W} ) ( 1 - \frac{{a}_{0}}{W} ) ( 2.15 - \frac{3.93 {a}_{0}}{W} + \frac{2.7 {a}_{0}^{2}}{{W}_{2}} ) ]}{2 ( 1 + \frac{2 {a}_{0}}{W} ) {( 1 - \frac{{a}_{0}}{W} )}^{1.5}}$$

$$δ = {[ \frac{FS}{{BW}^{1.5}} \times f ( \frac{{a}_{0}}{W} ) ]}^{2} \frac{( 1 - {v}^{2} )}{2 {σ}_{YS} E} + \frac{0.4 ( W - {a}_{0} ) {V}_{P}}{0.4 W + 0.6 {a}_{0} + z}$$

Wherein, F is load, S is span, W is width, B is thickness, a₀ is initial crack length, v is Poisson's ratio, σ_{YS} is yield strength, E is elastic modulus, V_{P} is plastic component of the notch opening displacement, Z is knife-edge thickness.

Optionally, the slow strain rate tensile test comprises:
stretching of the samples after thermal simulation to a specified strain in the air at a first preset stretching rate;
stretching of the samples after thermal simulation in a selected stretching solution at a second preset stretching rate at a preset tensile test temperature;
the stress corrosion cracking susceptibility parameters include the Sulfide Stress Corrosion Cracking (SSCC) sensitivity coefficient, which is calculated according to the formula:

$${S}_{ψ} = ( 1 - \frac{{ψ}_{s}}{{ψ}_{0}} ) \times 100 \% ,$$

wherein S_{ψ} is SSCC sensitivity coefficient, ψₛ is elongation in corrosive medium and ψ₀ is elongation in the air.

Optionally, the pre-strain sensitivity calculation formula is:

$$I = ( 1 - \frac{{ψ}_{p 1}}{{ψ}_{p 0}} ) \times 100 \% ,$$

wherein: ψₚ₀ is the elongation before pre-strain and ψₚ₁ is the elongation after pre-strain.

Optionally, the relationship between the secondary thermal simulation parameters and the welding heat input in the secondary control method is:

$$Q = \sqrt{\frac{4 π lpc Δ t}{\frac{1}{{( {T}_{2} - {T}_{0} )}^{2}} - \frac{1}{{( {T}_{1} - {T}_{0} )}^{2}}}} \cdot d$$

Wherein, Δt is target cooling time period, i.e. the secondary thermal simulation t_{8/5}. T₁ and T₂ are starting and ending temperatures of cooling, respectively, T₀ is preheating temperature, Q is welding heat input parameters, d is plate thickness, l is thermal conductivity, p is material density and c is specific heat capacity.

Optionally, the welding parameters are determined according to the welding heat input parameters, specifically comprising: using the following formula for calculation:

$$Q = IU η / V ;$$

wherein, Q is welding heat input parameters, I is welding current; U is arc voltage; V is the welding speed; η is welding thermal efficiency factor.

Optionally, the stress intensity factor of sulfur induced stress corrosion cracking is calculated as:

$${K}_{ISSC} = \frac{Pa ( 2 \sqrt{3} + 2.38 h / a ) {( B / {B}_{n} )}^{1 / \sqrt{3}}}{{Bh}^{3 / 2}}$$

Wherein, K_{ISSC} is stress intensity factor of sulfide stress corrosion cracking; P is load of balanced wedge block, measured values for loading surfaces; a is cracking length; h is the height of each cantilever; B is specimen thickness; and Bₙ is the web thickness.

Optionally, after determining the welding parameters according to the welding heat input parameters, it also includes:
welding according to welding parameters using CO₂ flux cored gas shielded welding to obtain test samples;
conducting CTOD tests, pre-strain tests and stress corrosion tests on test samples to obtain experimental results;
determining the final welding parameters by combining the experimental results.

Optionally, the experimental rate of CTOD experiment is 0.5 mm/min, the experimental temperature is −10° C., the experimental rate of pre-strain experiment is 0.5 mm/min, the experimental rate of stress corrosion experiment is 2×10⁻⁵ mm/s, and the experimental temperature is 23° C.

At least one of the above technical solutions adopted in the implementation of this manual can achieve the following beneficial effects:
(1) the present invention can determine the most ideal multi-layer and multi-pass welding heat input in a short time, without the need for actual welding with various parameters, which greatly reduces the time and cost required for designing and developing the welding process, and greatly improves the efficiency.
(2) the present invention conducts actual welding and testing after converting the optimal thermal simulation parameters into welding heat input to verify the accuracy of experimental results and ensure the safety of welded pipes in service.

### BRIEF DESCRIPTION OF THE DRAWINGS

The drawings described here are used to provide further understanding of this application and form part of this application. The indicative embodiment and its explanation are used to explain this application, which does not constitute an improper limitation of this application. In the drawings:

FIG. 1 provides a method for determining the filling welding parameters of large deformation pipeline steel based on the secondary control method for the embodiment of the invention;

FIG. 2 shows a schematic diagram of a CTOD specimen provided by an embodiment of the present invention;

FIG. 3 is the schematic diagram of the slow strain rate tensile specimen in the embodiment of the invention;

FIG. 4 is the schematic diagram of the double cantilever beam sample in the embodiment of the invention.

### DETAILED DESCRIPTION OF THE EMBODIMENTS

In order to make the purpose, technical solutions and advantages of the present application clearer, a clear and complete description of the technical solutions of the present application will be given below in connection with the specific embodiments of the present application and the corresponding accompanying drawings. Obviously, the described embodiments are only a part of the embodiments of the present application, and not all of them. Based on the embodiments in this application, all other embodiments obtained by a person of ordinary skill in the art without creative labour fall within the scope of protection of this application.

In multi-pass welding, the weld of the previous pass will be affected by the heat of the next pass, forming a complex heat-affected zone. The critical reheat coarse grain zone is the weakest area in the welded pipe, which has the lowest fracture toughness and SSCC is most likely to occur, affecting the safety of the pipeline in service. Therefore, the present invention provides a method for determining the multi-layer and multi-pass welding process parameters of X65 pipeline steel based on the secondary control method under the condition of pre-strain. The above problems are solved by developing and designing a welding process that realizes the synergistic improvement of sulfide stress corrosion cracking resistance and fracture toughness of the heat-affected zone of the pipeline steel under the action of pre-strain.

The Gleeble 3500 thermal simulation tester can accurately simulate the microstructure of the critical reheated coarse grain zone in the heat-affected zone under different heat inputs by varying the cooling rate under the premise of determining the heating rate and peak temperature. The base material is processed into a block specimen with a cross section of 10×10 mm² and a 2 mm thick plate specimen after the Gleeble3500 is used for thermal simulation of welding with different cooling rates. Firstly, fracture toughness tests and stress corrosion tests before and after pre-strain were carried out on thermally simulated specimens to determine the most desirable cooling rate. Then, the cooling rate is converted into welding heat input according to the thermal conductivity equation, and the process parameters are developed for carbon dioxide gas shielded welding, and the welded specimens are subjected to double cantilever beam experiments to verify the safety of the welding process.

The technical solutions provided by the various embodiments of the present application are described in detail below in conjunction with the accompanying drawings.

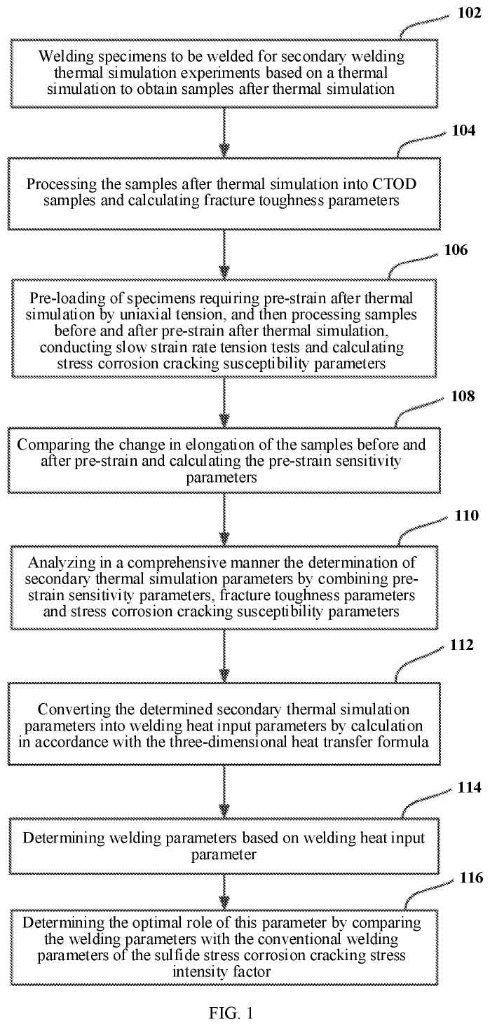

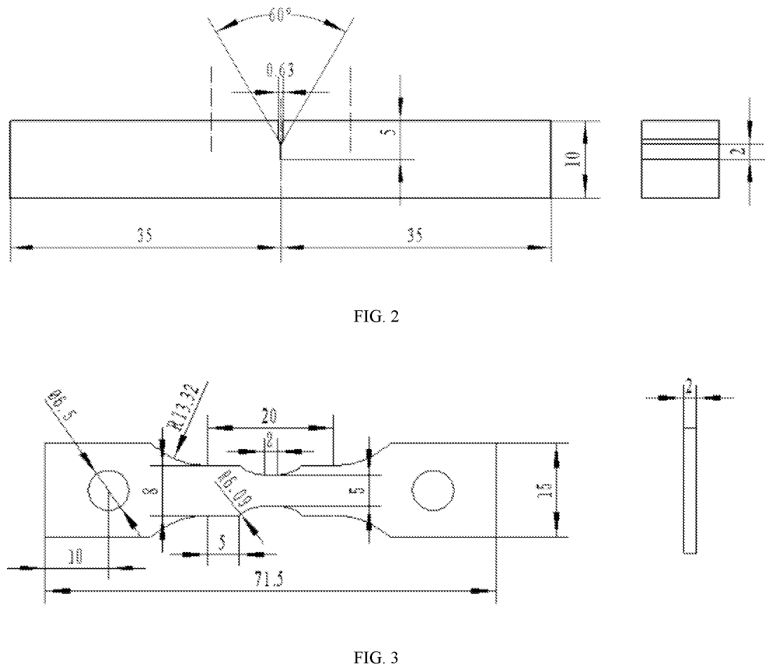

FIG. 1 provides a method for determining the filling welding parameters of large deformation pipeline steel based on the secondary control method for the embodiment of the invention. As shown in FIG. 1, the process may comprise the steps.
step 102: welding specimens to be welded for secondary welding thermal simulation experiments based on a thermal simulation to obtain samples after thermal simulation;
step 104: processing the samples after thermal simulation into CTOD samples and calculating fracture toughness parametersd;
step 106: pre-loading of specimens requiring pre-strain after thermal simulation by uniaxial tension, and then processing samples before and after pre-strain after thermal simulation, conducting slow strain rate tension tests and calculating stress corrosion cracking susceptibility parameters;
step 108: comparing the change in elongation of the samples before and after pre-strain and calculating the pre-strain sensitivity parameters;
step 110: analyzing in a comprehensive manner the determination of secondary thermal simulation parameters by combining pre-strain sensitivity parameters, fracture toughness parameters and stress corrosion cracking susceptibility parameters;
step 112: converting the determined the secondary thermal simulation parameters into welding beat input parameters by calculation in accordance with the three-dimensional heat transfer formula;
step 114: determining welding parameters based on welding heat input parameter;
step 116: determining the optimal role of this parameter by comparing the welding parameters with the conventional welding parameters of the sulfide stress corrosion cracking stress intensity factor.

Based on the method of FIG. 1, this manual example also provides some specific implementation methods of this method.

Step 1, welding specimens to be welded for welding thermal simulation experiments based on a thermal simulation to obtain samples after thermal simulation;

In the specific implementation, the selection of the base material is carried out firstly: selecting X65 pipe line steel as the base material, and the mechanical properties of the material are as follows:

| Material | Yield Strength(MPa) | Tensile Strength(MPa) |
|---|---|---|
| X65 | 501 | 588 |

Then the samples to be welded are processed: block samples with a cross section of 10×10mm² and plate samples with a thickness of 2 mm are processed along the rolling direction for thermal simulation. This example uses two different shapes of samples to be welded, so that a comprehensive performance test can be performed on the samples. However, some of the same or identical specimens to be welded can also be used, and the changes in the shape, size, material, and mechanical properties of the specimens to be welded are all within the protection range of the invention.

Then the second thermal simulation experiment is carried out. The specific thermal simulation experiment method can include but not be limited to the following steps:
a thermocouple wire is welded at the center of the sample to be welded to detect the relationship between temperature and time; a thermal simulation:
heating the specimen to 1350° C. at a rate of 130° C./s and holding for 1 s;
taking the cooling time period t_{8/5} (the time taken to cool from 800° C. to 500° C.) for 20 s cooling to 200° C.

Secondary thermal simulation:
heating the specimen to 750° C. at a rate of 130° C./s and holding for 1 s;
cooling to 900° C. at 80° C./s;
cooling time periods t_{8/5} are used for 20 s, 30 s, 40 s and 50 s to cool down to room temperature.

Step 2, processing the thermally simulated specimens into CTOD specimens by the CTOD experimental method and calculating the fracture toughness parameters;
CTOD experiment: processing the block specimens after thermal simulation into CTOD specimens with the specific dimensions shown in FIG. 3. The experimental rate is 0.5 mm/s and the experimental temperature is −10° C.;
fracture toughness calculation: the fracture of the specimen after the CTOD experiment is measured and the CTOD value is calculated to evaluate the fracture toughness at different t8/5 fracture toughness under different conditions.

Computation of the CTOD value δ is as follows:

$$f ( \frac{{a}_{0}}{W} ) = \frac{3 {( \frac{{a}_{0}}{W} )}^{0.5} [ 1.99 - ( \frac{{a}_{0}}{W} ) ( 1 - \frac{{a}_{0}}{W} ) ( 2.15 - \frac{3.93 {a}_{0}}{W} + \frac{2.7 {a}_{0}^{2}}{{W}^{2}} ) ]}{2 ( 1 + \frac{2 {a}_{0}}{W} ) {( 1 - \frac{{a}_{0}}{W} )}^{1.5}} \qquad ( 2 )$$

$$δ = {[ \frac{FS}{{BW}^{1.5}} \times f ( \frac{{a}_{0}}{W} ) ]}^{2} \frac{( 1 - {v}^{2} )}{2 {σ}_{YS} E} + \frac{0.4 ( W - {a}_{0} ) {V}_{P}}{0.4 W + 0.6 {a}_{0} + z} \qquad ( 3 )$$

Wherein: F is load, S is span, W is width, B is thickness, a₀ is initial crack length, v is Poisson's ratio, σ_{YS} is yield strength, E is elastic modulus, V_{P} is plastic component of the notch opening displacement, Z is knife-edge thickness. The following correspondence is obtained after calculation:

| t_{5/8} | 20 | 30 | 40 | 50 |
|---|---|---|---|---|
| δ (mm) | 0.0132 | 0.0095 | 0.0072 | 0.0028 |

Showing that the fracture toughness of the specimen decreases with increasing t_{8/5}.

Step 3, pre-straining or no manipulation of specimens after thermal simulation, then processing into slow strain rate tensile specimens for slow strain rate tensile tests and calculating corrosion cracking sensitivity parameters;
slow strain rate tensile test: the experimental temperature is room temperature, the second preset tensile rate is 2×10⁻⁵ mm/s, and the solution is a standard NACE A solution with 10⁻³ mol/L S₂O₃² added, that is, mass fraction of 5% NaCl and 0.5% CH₃COOH, the plate sample after thermal simulation is processed into a slow tensile sample, the specific size is shown in FIG. 3.

SSCC sensitivity calculation: the SSCC sensitivity of the sample is reflected by the elongation loss, and the optimal t_{8/5} is selected. The calculation of SSCC sensitivity coefficient (S_{ψ}) is shown in Formula (4):

$${S}_{ψ} = ( 1 - \frac{{ψ}_{s}}{{ψ}_{0}} ) \times 100 \% \qquad ( 4 )$$

Wherein, ψₛ is the elongation in corrosive medium, ψ₀ is the elongation in air.

Pre-strain sensitivity calculation: the pre-strain sensitivity of the sample is reflected by the loss of elongation before and after pre-strain, and the optimal t_{8/5} is selected. The calculation of pre-strain sensitivity coefficient (I) is shown in Formula (5):

$$I = ( 1 - \frac{{ψ}_{p 1}}{{ψ}_{p 0}} ) \times 100 \% \qquad ( 5 )$$

Wherein: ψₚ₀ is the elongation before pre-strain, ψₚ₁ is the elongation after pre-strain.

Step 4, welding heat input calculation: according to the three-dimensional heat transfer formula in the standard NACE SP0472-2010, the welding heat input is calculated.

$$Q = \sqrt{\frac{4 π lpc Δ t}{\frac{1}{{( {T}_{2} - {T}_{0} )}^{2}} - \frac{1}{{( {T}_{1} - {T}_{0} )}^{2}}}} \cdot d \qquad ( 6 )$$

Wherein, Δt is target cooling time period, i.e. the secondary thermal simulation t_{8/5}, T₁ and T₂ are starting and ending temperatures of cooling, respectively, T₀ is preheating temperature, Q is welding heat input parameters, d is plate thickness, l is thermal conductivity, p is material density and c is specific heat capacity.

After the welding heat input is obtained according to the above steps, the welding current (I) and arc voltage can be planned according to Formula (7).

Q=IUη/V (7)

Wherein, I is welding current; U is arc voltage; V is the welding speed; η is welding thermal efficiency factor.

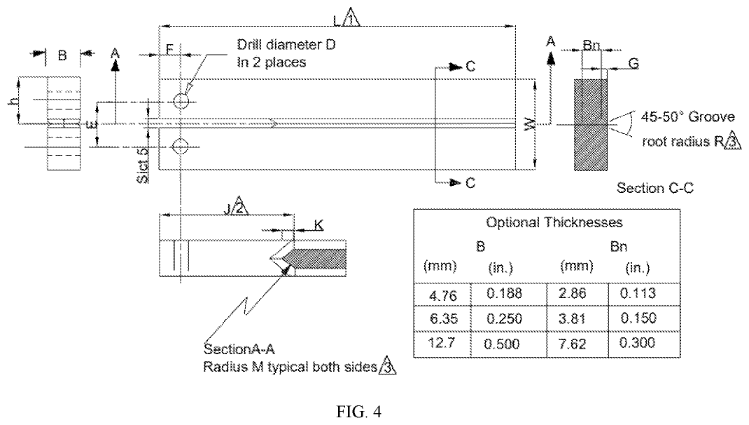

Step 5, the welded parts are tested for resistance to hydrogen sulfide stress corrosion: in accordance with the finalised welding parameters and traditional welding process parameters, the weld is welded using carbon dioxide gas shielded welding commonly used in industry, and the post-weld specimen is processed in accordance with the standard NACE TM 0177 as shown in FIG. 4, the specimen is measured and loaded with a wedge block and placed in NACEA solution, after being de-oxygenated by passing nitrogen and then injecting hydrogen sulfide gas, removing after 720 h soaking, the stress-strain curve is obtained by stretching using a tensile machine, and the critical stress intensity factor for sulfide stress corrosion is calculated.

$${K}_{ISSC} = \frac{Pa ( 2 \sqrt{3} + 2.38 h / a ) {( B / {B}_{n} )}^{1 / \sqrt{3}}}{{Bh}^{3 / 2}} \qquad ( 8 )$$

Wherein, K_{ISSC} is stress intensity factor of sulfide stress corrosion cracking; P is load of balanced wedge block, measured values for loading surfaces; a is cracking length; h is the height of each cantilever; B is specimen thickness; and Bₙ is the web thickness.

When the determined welding process KISSC is larger, it shows that the process can optimize and improve the weld performance compared with the traditional process, otherwise there is no optimization effect, so as to finally determine the welding process of X65 pipeline steel welded joint heat-affected zone against stress corrosion cracking.

After slow strain rate stretching and related calculations, the SSCC sensitivity coefficients and pre-strain sensitivity coefficients at different t_{8/5} are as follows:

| t [symbol] | | 20 | 30 | 40 | 50 |
|---|---|---|---|---|---|
| [symbol] (%) | pre-strain 0% | 88.86 | 87.79 | 87.37 | 87.19 |
| | pre-strain 5% | 88.83 | 87.51 | 87.15 | 86.09 |
| I (%) | In corrosive medium | 3.22 | 0.61 | 1.81 | 1.30 |
| | In the air | 4.48 | 0.84 | 3.15 | 7.14 |

[symbol]

indicates data missing or illegible when filed

When t_{8/5}=30 s, the SSCC sensitivity of the sample before and after pre-strain is low, and the pre-strain sensitivity is the lowest in different media, and the fracture toughness is also at a high level, indicating that the pre-strain is conducive to the synergistic improvement of fracture toughness and hydrogen sulfide stress corrosion cracking resistance. Then according to the three-dimensional heat transfer formula, the welding heat input Q can be obtained. According to its relationship with welding current, arc voltage and welding speed, a suitable multi-layer multi-pass welding process is designed and developed.

Step 6, through the double cantilever beam test of optimized parameters and traditional welding parameters, the strength coefficient of sulfur induced stress corrosion cracking is calculated, so as to verify the optimization effect of this parameter on pipeline welding performance.

$${K}_{ISSC} = \frac{Pa ( 2 \sqrt{3} + 2.38 h / a ) {( B / {B}_{n} )}^{1 / \sqrt{3}}}{{Bh}^{3 / 2}} \qquad ( 9 )$$

Wherein, K_{ISSC} is strength coefficient of sulfur induced stress corrosion cracking; P is load of balanced wedge block, measured values for loading surfaces; a is cracking length; h is the height of each cantilever; B is specimen thickness; and Bₙ is the web thickness.

It should be noted that the above steps 2 and 3 can be exchanged in order, or can be carried out at the same time. The selection of the target cooling time t_{8/5} can be comprehensively selected after obtaining three performance parameters, or can be selected step by step according to the specific experimental order. The order shown in this embodiment does not limit the invention, as long as the relevant steps containing the invention are within the protection range of the invention.

Another embodiment of the invention provides a method for welding using the welding parameters obtained by the above embodiment, which can include:
the welding process is determined according to the welding heat input and welding parameters determined by the above method;
welding is carried out according to the welding process.

The specific welding process determination method is the common knowledge in this field, which can be combined with the welding parameters according to the common knowledge in this field.

It is also important to note that the term ‘include’, ‘comprise’ or any other variant of the term is intended to cover non-exclusive inclusion, so that a process, method, commodity or device that includes a set of elements includes not only those elements but also other elements that are not explicitly listed or that are inherent to such a process, method, commodity or device. In the absence of more restrictions, by the statement ‘including a . . . The limited elements do not exclude that there are other same elements in the process, method, commodity or equipment including the elements.

The individual embodiments in this specification are described in a progressive manner, with the same similarities between the various embodiments being referred to each other, with each embodiment focusing on what is different from the others. In particular, for the system embodiment, which is essentially similar to the method embodiment, the description is simpler and the relevant parts are described in the method embodiment.

The above description is only an example of an embodiment of the present application and is not intended to limit it. For those skilled in the art The present application is subject to various modifications and variations. Any modifications, equivalents, replacements, improvements, etc., made within the spirit and principles of this application shall be included within the scope of this application.

What is claimed is:

1. A method for determining filling welding parameters of a large deformation pipeline steel based on a secondary regulation method, comprising:
welding specimens to be welded for secondary welding thermal simulation experiments based on a thermal simulation to obtain samples after thermal simulation;
processing the samples after thermal simulation into Crack-tip Opening Displacement (CTOD) samples and calculating fracture toughness parameters;
pre-loading of specimens requiring pre-strain after thermal simulation by uniaxial tension, then processing samples before and after pre-strain after thermal simulation, conducting slow strain rate tension tests, and calculating stress corrosion cracking susceptibility parameters;
comparing a change in elongation of the samples before and after pre-strain and calculating pre-strain sensitivity parameters;
analyzing, in a comprehensive manner, determination of secondary thermal simulation parameters by combining the pre-strain sensitivity parameters, the fracture toughness parameters and the stress corrosion cracking susceptibility parameters;
converting the secondary thermal simulation parameters into welding heat input parameters by calculation in accordance with a three-dimensional heat transfer formula;
determining welding parameters based on the welding heat input parameters; and
determining an optimal role of the welding parameters by comparing the welding parameters with conventional welding parameters of a sulfide stress corrosion cracking stress intensity factor.

2. The method according to claim 1, wherein the samples to be welded are multiple, and multiple samples to be welded have different cooling rates for a secondary heat cycle.

3. The method according to claim 1, wherein the fracture toughness parameters comprise a CTOD value, and a calculation formula of the fracture toughness parameters is:
$$f ( \frac{{a}_{0}}{W} ) = \frac{3 {( \frac{{a}_{0}}{W} )}^{0.5} [ 1.99 - ( \frac{{a}_{0}}{W} ) ( 1 - \frac{{a}_{0}}{W} ) ( 2.15 - \frac{3.93 {a}_{0}}{W} + \frac{2.7 {a}_{0}^{2}}{{W}^{2}} ) ]}{2 ( 1 + \frac{2 {a}_{0}}{W} ) {( 1 - \frac{{a}_{0}}{W} )}^{1.5}} \\ δ = {[ \frac{FS}{{BW}^{1.5}} \times f ( \frac{{a}_{0}}{W} ) ]}^{2} \frac{( 1 - {v}^{2} )}{2 {σ}_{YS} E} + \frac{0.4 ( W - {a}_{0} ) {V}_{P}}{0.4 W + 0.6 {a}_{0} + z}$$
wherein, F is load, S is span, W is width, B is thickness, a₀ is initial crack length, v is Poisson's ratio, σ_{YS} is yield strength, E is elastic modulus, V_{P} is plastic component of a notch opening displacement, Z is knife-edge thickness.

4. The method according to claim 1, wherein a slow tensile test comprises:
stretching the samples after thermal simulation to a specified strain in air at a first preset stretching rate; and
stretching the samples after thermal simulation in a selected stretching solution at a second preset stretching rate at a preset tensile test temperature;
wherein the stress corrosion cracking susceptibility parameters comprise a Sulfide Stress Corrosion Cracking (SSCC) sensitivity coefficient, wherein the SSCC sensitivity coefficient is calculated according to the formula:
$${S}_{ψ} = ( 1 - \frac{{ψ}_{s}}{{ψ}_{0}} ) \times 100 \% ,$$
wherein S_{ψ} is SSCC sensitivity coefficient, ψₛ is elongation in corrosive medium, and ψ₀ is elongation in the air.

5. The method according to claim 1, wherein a pre-strain sensitivity calculation equation is:
$$I = ( 1 - \frac{{ψ}_{p 1}}{{ψ}_{p 0}} ) \times 100 \%$$
wherein ψₚ₀ is an elongation before pre-strain, and ψₚ₁ is an elongation after pre-strain.

6. The method according to claim 1, wherein a relationship between the secondary thermal simulation parameters and the welding heat input parameters in the secondary regulation method is:
$$Q = \sqrt{\frac{4 π lpc Δ t}{\frac{1}{{( {T}_{2} - {T}_{0} )}^{2}} - \frac{1}{{( {T}_{1} - {T}_{0} )}^{2}}}} \cdot d ;$$
wherein Δt is target cooling time period, i.e. the secondary thermal simulation t_{8/5}, T₁ and T₂ are starting and ending temperatures of cooling, respectively, T₀ is preheating temperature, Q is the welding heat input parameters, d is plate thickness, l is thermal conductivity, p is material density, and c is specific heat capacity.

7. The method according to claim 1, wherein the welding parameters are determined according to the welding heat input parameters, specifically comprising: using the following formula for calculation:
$$Q = IU η / V ;$$
wherein Q is the welding heat input parameters, I is welding current, U is arc voltage, V is welding speed, and η is welding thermal efficiency factor.

8. The method according to claim 1, wherein the sulfide stress corrosion cracking stress intensity factor is calculated as:
$${K}_{ISSC} = \frac{Pa ( 2 \sqrt{3} + 2.38 h / a ) {( B / {B}_{n} )}^{1 / \sqrt{3}}}{{Bh}^{3 / 2}} ;$$
wherein K_{ISSC} is the sulfide stress corrosion cracking stress intensity factor; P is load of balanced wedge block, measured values for loading surfaces; a is cracking length; h is the height of each cantilever; B is specimen thickness; and Bₙ is the web thickness.

9. The method according to claim 1, wherein after determining the welding parameters based on the welding heat input parameters, further comprising:
welding according to welding parameters using CO₂ flux cored gas shielded welding to obtain test samples;
conducting CTOD tests, pre-strain tests and stress corrosion tests on the test samples to obtain experimental results; and
determining the final welding parameters by combining the experimental results.

10. The method according to claim 9, wherein an experimental rate of the CTOD tests is 0.5 mm/min, an experimental temperature is −10° C.; an experimental rate of the pre-strain tests is 0.5 mm/min; an experimental rate of the stress corrosion tests is 2×10⁻⁵ mm/s, and an experimental temperature is 23° C.
